(19) United States
(12) Patent Application Publication
(10) Pub. No.: US 2025/0098944 A1
(43) Pub. Date: Mar. 27, 2025

(54) ENDOSCOPE

(71) Applicant: OLYMPUS MEDICAL SYSTEMS CORP., Tokyo (JP)

(72) Inventor: Yuichi Wataya, Akiruno-shi (JP)

(73) Assignee: OLYMPUS MEDICAL SYSTEMS CORP., Tokyo (JP)

(21) Appl. No.: 18/895,883

(22) Filed: Sep. 25, 2024

Related U.S. Application Data
(60) Provisional application No. 63/540,408, filed on Sep. 26, 2023.

(51) Int. Cl. A61B 1/00; A61B 1/05

(52) U.S. Cl. CPC A61B 1/00193; A61B 1/00131; A61B 1/05

(57) ABSTRACT

What is provided is an endoscope capable of improving the quality of a stereoscopic image of a subject.

The endoscope includes a lens frame configured to hold each of a first objective optical system and a second objective optical system and to extend in an optical axis direction, an imaging unit having an imaging element, and a heat dissipation member in contact with each of the lens frame and the imaging unit.

The present invention relates to an endoscope. Priority is claimed on U.S. Provisional Application No. 63/540,408, filed Sep. 26, 2023, the content of which is incorporated herein by reference.

### BACKGROUND OF THE INVENTION

### Field of the Invention

### Description of Related Art

In recent years, there has been an increasing demand for medical endoscopes and industrial endoscopes that enable stereoscopic observation of a subject. In response to such a demand, an endoscope that includes two objective optical systems and two imaging elements that receive optical images of the subject formed by each of the two objective optical systems is known (for example, Patent Document 1).

### PATENT DOCUMENTS

[Patent Document 1] Japanese Patent Publication No. 6188988

### SUMMARY OF THE INVENTION

In the above-described endoscope, the two imaging elements may be disposed close to each other in order to reduce a size of the endoscope. In this case, heat generated in each of the imaging elements is difficult to dissipate, and thus a temperature of each of the imaging elements is likely to become high. When the temperature of each of the imaging elements becomes too high, dark current noise in each of the imaging elements increases, and thus an image signal generated in each of the imaging elements deteriorates. Therefore, there is a risk that the quality of a stereoscopic image of the subject observed by a user of the endoscope may be degraded.

In view of the above circumstances, an object of the present invention is to provide an endoscope capable of improving the quality of a stereoscopic image of a subject.

In order to achieve the above object, an endoscope according to one aspect of the present invention includes a lens frame configured to hold each of a first objective optical system and a second objective optical system and to extend in an optical axis direction, an imaging unit having an imaging element, and a heat dissipation member in contact with each of the lens frame and the imaging unit.

According to the present invention, an endoscope capable of improving the quality of a stereoscopic image of a subject can be provided.

### BRIEF DESCRIPTION OF THE DRAWINGS

FIG. 1 is a perspective view showing an endoscope system according to a first embodiment.

FIG. 2 is an external view of an optical unit of the first embodiment when seen from a first side in a second direction.

FIG. 3 is a cross-sectional view showing the optical unit of the first embodiment and taken along line III-III in FIG. 2.

FIG. 4 is a cross-sectional view showing the optical unit of the first embodiment and taken along line IV-IV in FIG. 3.

FIG. 5 is a cross-sectional view showing an optical unit according to a first modified example of the first embodiment.

FIG. 6 is a cross-sectional view showing an optical unit according to a second modified example of the first embodiment.

FIG. 7 is a cross-sectional view showing an optical unit according to a third modified example of the first embodiment.

FIG. 8 is a cross-sectional view showing an optical unit according to a fourth modified example of the first embodiment.

FIG. 9 is an external view of an optical unit according to a second embodiment when seen from a second side in the second direction.

FIG. 10 is a cross-sectional view showing the optical unit according to the second embodiment and taken along line X-X in FIG. 9.

FIG. 11 is a cross-sectional view showing the optical unit according to the second embodiment and taken along line XI-XI in FIG. 10.

FIG. 12 is an external view of an optical unit according to a third embodiment when seen from a first side in the second direction.

FIG. 13 is a cross-sectional view showing the optical unit according to the third embodiment and taken along line XIII-XIII in FIG. 12.

FIG. 14 is an external view of an optical unit according to a fourth embodiment when seen from a first side in the second direction.

### DETAILED DESCRIPTION OF THE INVENTION

Hereinafter, an endoscope according to an embodiment of the present invention will be described with reference to the drawings. The scope of the present invention is not limited to the following embodiments, and can be arbitrarily modified within the scope of the technical concept of the present invention. In addition, in the drawings below, the scale and number of each structure may differ from the actual structure in order to make each configuration easier to understand.

In each drawing, a Z axis is appropriately indicated. The Z axis is a direction in which a first axis J1 and a second axis J2 in the embodiment described below extend. The first axis J1 and the second axis J2 shown in each drawing are virtual axes. The first axis J1 is a central axis of a first lens frame. The second axis J2 is a central axis of a second lens frame. In the following description, the direction in which the first axis J1 and the second axis J2 extend, that is, the direction parallel to the Z axis, will be referred to as an “optical axis direction.” The side (the +Z side) in the optical axis direction toward which a Z-axis arrow points is the “object side (the distal side),” and the side in the optical axis direction opposite to the side toward which the Z-axis arrow points (−Z side) is the “base end side (the proximal side).”

A first direction D1 shown in each drawing is a direction that intersects with the optical axis direction. In this embodiment, the first direction D1 is a direction perpendicular to the optical axis direction. The first direction D1 may not be the direction perpendicular to the optical axis direction. In the following description, the side (the +D1 side) toward which an arrow of the first direction D1 points will be referred to as “a first side of the first direction D1,” and the side (the −D1 side) opposite to the side toward which the arrow of the first direction D1 points will be referred to as “a second side of the first direction D1.”

A second direction D2 which is appropriately shown in each drawing is a direction perpendicular to both the optical axis direction and the first direction D1. In the following description, the side (the +D2 side) toward which an arrow of the second direction D2 points will be referred to as “a first side of the second direction D2,” and the side (the −D2 side) opposite to the side toward which the arrow of the second direction D2 points will be referred to as “a second side of the second direction D2.”

In the following description, inward in the second direction D2 is a direction that faces a first virtual line in the second direction D2 when seen in the optical axis direction. Further, outward in the second direction D2 is a direction that faces the side opposite to the direction facing the first virtual line in the second direction D2 when seen in the optical axis direction.

### First Embodiment

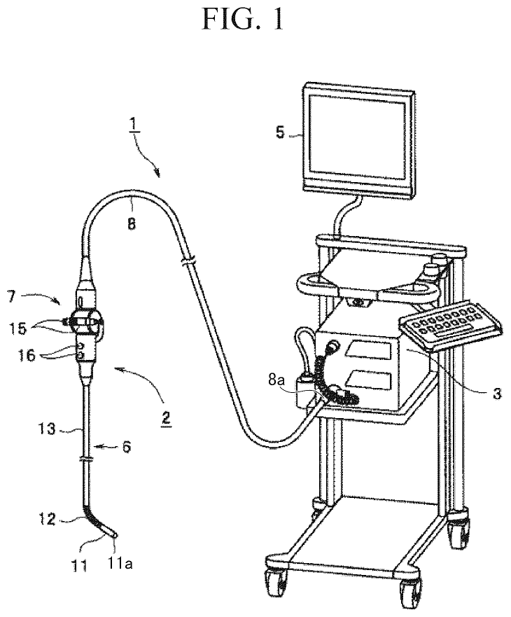

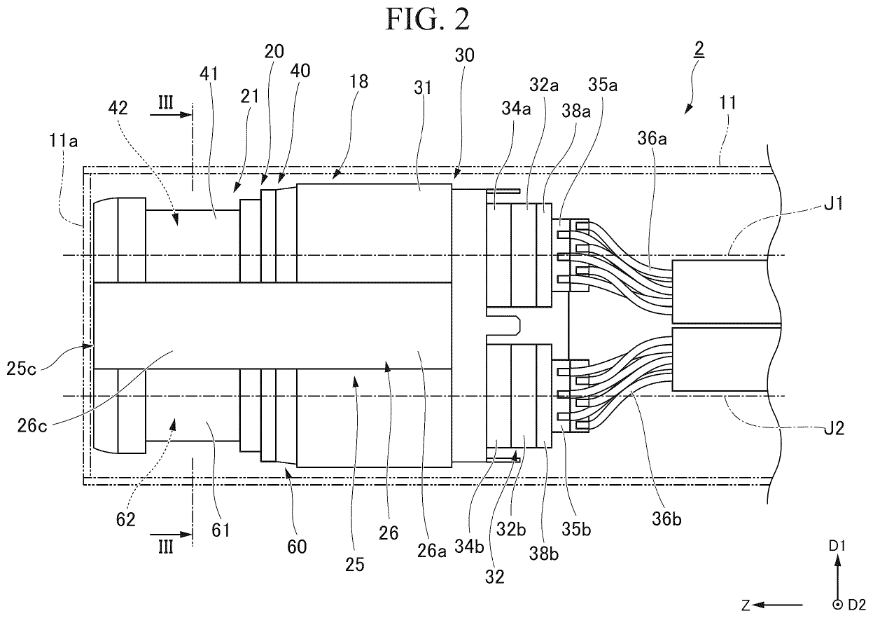

FIG. 1 is a perspective view showing an endoscope system 1 according to this embodiment. FIG. 2 is an external view of an optical unit 18 according to this embodiment when seen from a first side (the +D2 side) in the second direction D2.

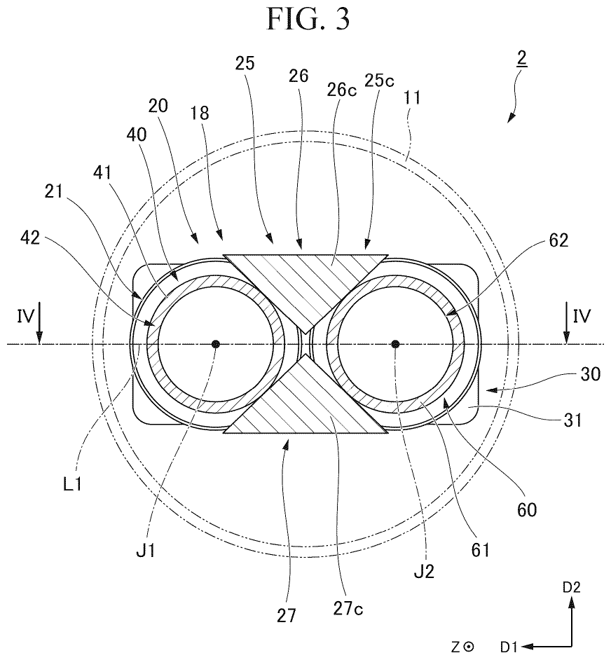

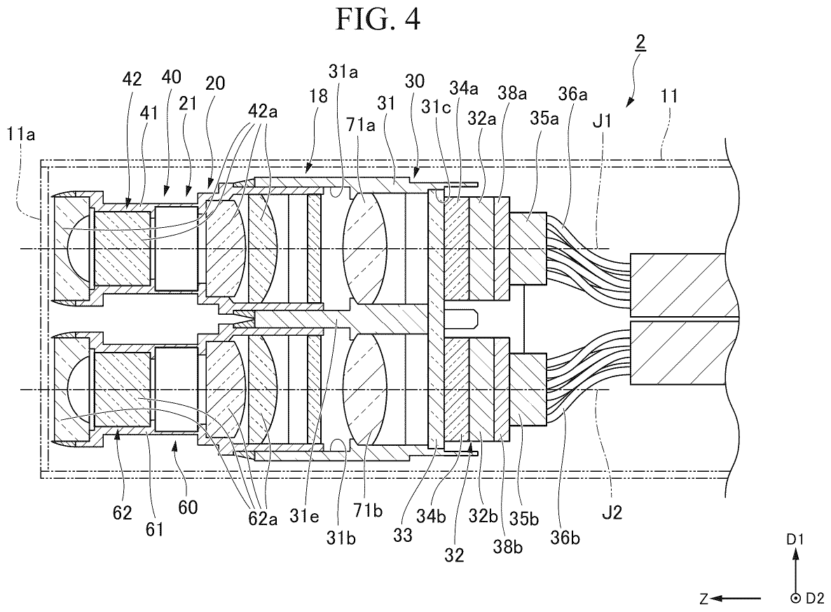

FIG. 3 is a cross-sectional view showing the optical unit 18 and taken along line III-III in FIG. 2. FIG. 4 is a cross-sectional view showing the optical unit 18 and taken along line IV-IV in FIG. 3.

The endoscope system 1 shown in FIG. 1 includes an endoscope 2 capable of stereoscopically capturing an image of a subject from different viewpoints and generating a stereoscopic image of the subject, a processor 3 to which the endoscope 2 is detachably connected, and a monitor 5 that displays an image signal generated by the processor 3 as the image of the subject.

The endoscope 2 includes an elongated insertion part 6, an operation part 7 connected to the base end side of the insertion part 6, and a universal cable 8 that extends from the operation part 7 and is connected to the processor 3. The use of the endoscope 2 of this embodiment is not particularly limited, and may be, for example, a medical endoscope or an industrial endoscope.

The insertion part 6 is a portion that is inserted, for example, into a body of a subject when the subject is observed. The insertion part 6 is configured by sequentially arranging a tip end portion 11, a bending portion 12, and a rigid tube portion 13 in this order from the object side, which is a tip end in a direction of insertion into the subject, toward the base end side connected to the operation part 7. The tip end portion 11 and the rigid tube portion 13 are made of a metal tube such as stainless steel, or the like. As shown in FIG. 2, in this embodiment, the tip end portion 11 has a substantially cylindrical shape that extends in the optical axis direction. The tip end portion 11 has a tip end surface 11a. The tip end surface 11a is a surface of an outer surface of the tip end portion 11 that faces the object side (the +Z side). The tip end surface 11a is provided with a well-known tip end opening, an observation window, and an illumination window (not shown). The optical unit 18 for stereoscopically capturing an image of the subject is disposed inside the tip end portion 11. The optical unit 18 is disposed at a position at which it faces the observation window (not shown). Therefore, the optical unit 18 can capture an image of the subject through the observation window.

Inside the bending portion 12 and the rigid tube portion 13 shown in FIG. 1, imaging cable bundles 36a and 36b (refer to FIG. 2, and the like) that electrically connect the optical unit 18 to the operation part 7 and the processor 3, and a light guide bundle (not shown) that transmits illumination light to the tip end portion 11 are inserted. The endoscope 2 of this embodiment is a rigid endoscope in which the rigid tube portion 13 is disposed closer to the base end side than the bending portion 12, but is not limited thereto, and the endoscope 2 may also be a flexible endoscope in which a flexible tube portion with flexibility is disposed closer to the base end side than the bending portion 12.

The operation part 7 is provided with an angle lever 15 that remotely controls the bending portion 12, a light source device (not shown) provided in the processor 3, and various switches 16 that adjusts a focal position of an optical image of the subject formed by the optical unit 18. The angle lever 15 is an operating means for operating the bending portion 12 to bend in four directions, that is, upward, downward, leftward, and rightward. The bending portion 12 is not limited to being bendable in the four directions, upward, downward, leftward, and rightward, and may be bendable in only the two directions, upward and downward, or only leftward and rightward, for example.

Inside the universal cable 8, the imaging cable bundles 36a and 36b (refer to FIG. 2 and the like) and the light guide bundle (not shown) and the like are inserted. A connector part 8a that is connected to the processor 3 is provided at an end portion of the universal cable 8 on the base end side. When the connector part 8a is connected to the processor 3, electrical components such as an imaging element of the endoscope 2 are communicatively connected to the processor 3 via the imaging cable bundles 36a and 36b.

As shown in FIG. 2, the optical unit 18 is disposed inside the tip end portion 11. The optical unit 18 forms an optical image of the subject, converts the optical image of the subject into an image signal, and transmits the image signal to the processor 3. The optical unit 18 includes an objective lens unit 20, a heat dissipation member 25, an imaging unit 30, and heat transfer members 38a and 38b. That is, the endoscope 2 includes the heat dissipation member 25, the imaging unit 30, and the heat transfer members 38a and 38b.

The objective lens unit 20 forms an optical image of the subject. The objective lens unit 20 is a part of the optical unit 18 on the objective side (the +Z side). The objective lens unit 20 includes a first objective lens unit 40 and a second objective lens unit 60. Each of the first objective lens unit 40 and the second objective lens unit 60 has a substantially cylindrical shape that extends in the optical axis direction. As shown in FIG. 3, the first objective lens unit 40 and the second objective lens unit 60 are disposed with an interval from each other in the first direction D1. The first objective lens unit 40 is disposed on a first side (the +D1 side) of the second objective lens unit 60 in the first direction D1.

As shown in FIG. 2, the objective lens unit 20 includes a lens frame 21. That is, the endoscope 2 includes the lens frame 21. The lens frame 21 holds the objective optical systems 42 and 62. The lens frame 21 includes a first lens frame 41 and a second lens frame 61. In this embodiment, the lens frame 21 is made of a metal such as stainless steel. The lens frame 21 may be made of other materials such as a resin material or ceramic.

As shown in FIG. 4, the first objective lens unit 40 includes the first lens frame 41 and the first objective optical system 42. The first lens frame 41 has a substantially cylindrical shape that extends in the optical axis direction with the first axis J1 as a center thereof. The first lens frame 41 may have another shape such as a prismatic shape that extends in the optical axis direction. The first lens frame 41 is open on both sides in the optical axis direction.

The first objective optical system 42 forms an optical image of the subject. An optical axis of the first objective optical system 42 in this embodiment overlaps the first axis J1 when seen in the optical axis direction. The optical axis of the first objective optical system 42 does not have to overlap the first axis J1 when seen in the optical axis direction. The first objective optical system 42 is disposed inside the first lens frame 41. The first objective optical system 42 is configured of a plurality of lenses 42a disposed in the optical axis direction. Each of the plurality of lenses 42a is held on an inner circumferential surface of the first lens frame 41. Thus, the first lens frame 41 holds the first objective optical system 42. That is, the lens frame 21 holds the first objective optical system 42.

The second objective lens unit 60 includes the second lens frame 61 and the second objective optical system 62. The second lens frame 61 has a substantially cylindrical shape that extends in the optical axis direction with the second axis J2 as a center thereof. The second lens frame 61 may have another shape such as a prismatic shape that extends in the optical axis direction. The second lens frame 61 is open on both sides in the optical axis direction. The shape of the second lens frame 61 is substantially the same as the shape of the first lens frame 41. As shown in FIG. 3, the second lens frame 61 is disposed closer to the second side (the −D1 side) in the first direction D1 than the first lens frame 41. In this embodiment, the first lens frame 41 and the second lens frame 61 are disposed with an interval therebetween in the first direction D1.

The second objective optical system 62 forms an optical image of the subject. As shown in FIG. 4, the optical axis of the second objective optical system 62 of this embodiment overlaps the second axis J2 when seen in the optical axis direction. The optical axis of the second objective optical system 62 does not have to overlap the second axis J2 when seen in the optical axis direction. The second objective optical system 62 is disposed inside the second lens frame 61. The second objective optical system 62 is configured of a plurality of lenses 62a disposed in the optical axis direction. Each of the plurality of lenses 62a is held on an inner circumferential surface of the second lens frame 61. Thus, the second lens frame 61 holds the second objective optical system 62. That is, the lens frame 21 holds the second objective optical system 62. As described above, the first lens frame 41 and the second lens frame 61 are disposed in the first direction D1.

As shown in FIG. 4, the imaging unit 30 is disposed inside the tip end portion 11. The imaging unit 30 is disposed closer to the base end side (the −Z side) than the objective lens unit 20. The imaging unit 30 has an imaging element holding part 31, an imaging element 32, a glass member 33, two cover glasses 34a and 34b, and two circuit boards 35a and 35b.

The imaging element holding part 31 has a generally rectangular tubular shape that extends in the optical axis direction. As shown in FIG. 3, when seen in the optical axis direction, the imaging element holding part 31 has a rectangular shape with rounded corners of which long sides extend in the first direction D1. In this embodiment, the imaging element holding part 31 is made of a resin. As shown in FIG. 4, the imaging element holding part 31 has a first holding hole 31a, a second holding hole 31b, a recessed portion 31c, and a partition wall portion 31e.

Each of the first holding hole 31a and the second holding hole 31b is a hole that passes through the imaging element holding part 31 in the optical axis direction. The first holding hole 31a is a substantially circular hole with the first axis J1 as a center thereof. The second holding hole 31b is a substantially circular hole with the second axis J2 as a center thereof. The partition wall portion 31e is a portion of the imaging element holding part 31 between the first holding hole 31a and the second holding hole 31b. The inside of the first holding hole 31a and the inside of the second holding hole 31b are separated by the partition wall portion 31e.

A portion of the first lens frame 41 on the base end side (the −Z side) is inserted into a portion of the first holding hole 31a on the object side (the +Z side). The portion of the first lens frame 41 on base end side is fixed to an inner circumferential surface of the first holding hole 31a. Thus, the imaging element holding part 31 holds the first objective lens unit 40. In this embodiment, the inner circumferential surface of the first holding hole 31a and the first lens frame 41 are fixed by adhesive. The inner circumferential surface of the first holding hole 31a and the first lens frame 41 may be fixed by other methods such as press fitting. A lens 71a is accommodated in a portion of the first holding hole 31a on the base end side. The lens 71a is held on the inner circumferential surface of the first holding hole 31a. The optical image of the subject that has passed through the first objective optical system 42 passes through the lens 71a to the base end side.

A portion of the second lens frame 61 on the base end side (the −Z side) is inserted into a portion of the second holding hole 31b on the object side (the +Z side).

The portion of the second lens frame 61 on the base end side is fixed to the inner circumferential surface of the second holding hole 31b. Thus, the imaging element holding part 31 holds the second objective lens unit 60. In this embodiment, the inner circumferential surface of the second holding hole 31b and the second lens frame 61 are fixed by adhesive. The inner circumferential surface of the second holding hole 31b and the second lens frame 61 may be fixed by other methods such as press fitting. A lens 71b is accommodated in a portion of the second holding hole 31b on the base end side. The lens 71b is held on the inner circumferential surface of the second holding hole 31b. The optical image of the subject that has passed through the second objective optical system 62 passes through the lens 71b to the base end side.

The recessed portion 31c is a hole recessed from a surface of the imaging element holding part 31 that faces the base end side (the −Z side) toward the object side (the +Z side). When seen in the optical axis direction, the recessed portion 31c overlaps the first holding hole 31a and the second holding hole 31b. The inside of the recessed portion 31c is connected to both the first holding hole 31a and the second holding hole 31b.

The glass member 33 has a plate shape that extends in a direction perpendicular to the optical axis direction. The glass member 33 is a transparent glass substrate. The glass member 33 is fixed to an inner surface of the recessed portion 31c by adhesive. The glass member 33 is disposed closer to the base end side (the −Z side) than the lenses 71a and 71b.

Each of the cover glasses 34a and 34b has a plate shape that extends in the direction perpendicular to the optical axis direction. Each of the cover glasses 34a and 34b is a transparent glass board. Each of the cover glasses 34a and 34b is fixed to a surface of the glass member 33 that faces the base end side (the −Z side). When seen in the optical axis direction, the cover glass 34a overlaps the first objective optical system 42 and the lens 71a. When seen in the optical axis direction, the cover glass 34b overlaps the second objective optical system 62 and the lens 71b.

The imaging element 32 converts the optical image of the subject formed by the objective lens unit 20 into an image signal. The imaging element 32 is, for example, an image sensor such as a CCD or a CMOS. The imaging element 32 is disposed closer to the base end side (the −Z side) than the cover glasses 34a and 34b. In this embodiment, the imaging element 32 includes a first imaging element 32a and a second imaging element 32b. That is, the imaging unit 30 has at least one imaging element 32.

The first imaging element 32a receives an optical image of the subject formed by the first objective optical system 42 and converts it into an image signal. A light receiving surface of the first imaging element 32a is fixed to the cover glass 34a. The second imaging element 32b receives an optical image of the subject formed by the second objective optical system 62 and converts it into an image signal. A light receiving surface of the second imaging element 32b is fixed to the cover glass 34b. The first imaging element 32a and the second imaging element 32b are held by the imaging element holding part 31 via the glass member 33 and the cover glasses 34a and 34b. That is, the imaging element holding part 31 holds the imaging element 32.

The number of imaging elements 32 included in the imaging unit 30 is not limited to two, but may be one, or three or more. For example, when the imaging unit 30 includes one imaging element 32, the light receiving surface of the imaging element 32 is fixed to each of the cover glasses 34a and 34b. Thus, the single imaging element 32 can receive the optical images of the subject formed by the first objective optical system 42 and the second objective optical system 62, and can convert each of the optical images of the subject into an image signal. Furthermore, when the imaging unit 30 includes three or more imaging elements 32, resolution and dynamic range of a stereoscopic image of the subject can be improved compared to when the imaging unit 30 includes two imaging elements 32. Therefore, the quality of the stereoscopic image of the subject can be improved more suitably.

As shown in FIG. 2, each of the circuit boards 35a and 35b is electrically connected to the imaging element 32. More specifically, the circuit board 35a is electrically connected to the first imaging element 32a, and the circuit board 35b is electrically connected to the second imaging element 32b. Each of the circuit boards 35a and 35b is, for example, a flexible printed circuit board (FPC board). Electronic components such as a digital IC that generates a drive signal for the imaging element 32 and a capacitor that stabilizes a drive power supply for the digital IC are mounted on each of the circuit boards 35a and 35b. The imaging cable bundle 36a is connected to the circuit board 35a, and the imaging cable bundle 36b is connected to the circuit board 35b. Thus, the imaging element 32 and the processor 3 (refer to FIG. 1) are connected so as to be able to communicate with each other.

As shown in FIG. 4, the first axis J1 passes through the first objective optical system 42, the lens 71a, the glass member 33, the cover glass 34a, and the first imaging element 32a. The optical image of the subject formed by the first objective optical system 42 passes through the lens 71a, the glass member 33, and the cover glass 34a, is received by the first imaging element 32a, and is converted into an image signal. The second axis J2 passes through the second objective optical system 62, the lens 71b, the glass member 33, the cover glass 34b, and the second imaging element 32b. The optical image of the subject formed by the second objective optical system 62 passes through the lens 71b, the glass member 33, and the cover glass 34b, and is converted into an image signal by the second imaging element 32b. The image signals converted by the first imaging element 32a and the second imaging element 32b are transmitted to the processor 3 via the circuit boards 35a and 35b and the imaging cable bundles 36a and 36b, and a stereoscopic image of the subject is formed in the processor 3.

Each of the heat transfer members 38a and 38b dissipates heat generated in the imaging element 32. Each of the heat transfer members 38a and 38b is disposed closer to the base end side (the −Z side) than the imaging element 32. Each of the heat transfer members 38a and 38b is made of a metal such as aluminum, copper, or the like. Each of the heat transfer members 38a and 38b has a higher thermal conductivity than the imaging element holding part 31. The heat transfer member 38a is in contact with the first imaging element 32a. The heat transfer member 38b is in contact with the second imaging element 32b. In other words, the heat transfer members 38a and 38b are in contact with the imaging unit 30. Heat generated in each of the imaging elements 32a and 32b is transferred to the heat transfer members 38a and 38b and dissipated into the atmosphere inside the tip end portion 11. Each of the heat transfer members 38a and 38b may be connected to the tip end portion 11 via a heat transfer member made of, for example, a metal. In this case, since the heat generated in each of the imaging elements 32a and 32b can be dissipated to the outside of the endoscope 2 via the heat transfer members 38a and 38b, the heat transfer member, and the tip end portion 11, it is possible to more effectively limit temperatures of the imaging elements 32a and 32b from becoming too high.

The heat dissipation member 25 transfers the heat generated in the imaging element 32 to the lens frame 21. As shown in FIG. 2, the heat dissipation member 25 extends in the optical axis direction. When seen in the second direction D2, the heat dissipation member 25 overlaps both the objective lens unit 20 and the imaging unit 30. In this embodiment, the heat dissipation member 25 is made of a metal. The heat dissipation member 25 can be made of, for example, aluminum, copper, silver, or an alloy containing these metals. As shown in FIG. 3, the heat dissipation member 25 has a contact portion 25c that is in contact with the lens frame 21. The contact portion 25c includes a first contact portion 26c and a second contact portion 27c which will be described below. In this embodiment, the endoscope 2 includes two heat dissipation members 25. The heat dissipation member 25 includes a first heat dissipation member 26 and a second heat dissipation member 27. A first virtual line L1 shown in FIG. 3 is a straight line that intersects with each of the first axis J1 which is the central axis of the first lens frame 41 and the second axis J2 which is the central axis of the second lens frame 61, and extends in the first direction D1. The first virtual line L1 is a virtual straight line. The first heat dissipation member 26 is disposed closer to a first side (the +D2 side) in the second direction D2 than the first virtual line L1. The second heat dissipation member 27 is disposed closer to a second side (the −D2 side) in the second direction D2 than the first virtual line L1.

As shown in FIG. 2, the first heat dissipation member 26 is disposed closer to first side (the +D2 side) in the second direction D2 than the objective lens unit 20 and the imaging unit 30. The first heat dissipation member 26 extends in the optical axis direction. When seen in the second direction D2, the first heat dissipation member 26 has a substantially rectangular shape of which long sides extend in the optical axis direction. The first heat dissipation member 26 has a first fixing portion 26a and a first contact portion 26c.

The first fixing portion 26a has a plate shape that extends in the optical axis direction. The first fixing portion 26a is a portion of the first heat dissipation member 26 on the base end side (the −Z side). The first fixing portion 26a is fixed by adhesion to a surface of the imaging element holding part 31 that faces a first side (the +D2 side) in the second direction D2. Thus, the first heat dissipation member 26 is in contact with the imaging element holding part 31. That is, the heat dissipation member 25 is in contact with the imaging element holding part 31. Therefore, the heat dissipation member 25 is in contact with the imaging unit 30.

The first contact portion 26c has a triangular prism shape that extends from an end portion of the first fixing portion 26a on the object side (the +Z side) toward the object side. In this embodiment, an end portion of the first contact portion 26c on the object side is located closer to the base end side (the −Z side) than an end portion of the objective lens unit 20 on the object side. As shown in FIG. 3, when seen in the optical axis direction, the first contact portion 26c has a substantially triangular shape that protrudes toward the first virtual line L1. In the second direction D2, a dimension of a portion of the first contact portion 26c in the first direction D1 that is farthest from the first virtual line L1 in the second direction D2 is larger than a dimension of a portion of the first contact portion 26c in the first direction D1 that is closest to the first virtual line L1. A part of the first contact portion 26c is disposed between the first lens frame 41 and the second lens frame 61, and is in contact with both the first lens frame 41 and the second lens frame 61. That is, at least a part of the heat dissipation member 25 is disposed between the first lens frame 41 and the second lens frame 61 and is in contact with both the first lens frame 41 and the second lens frame 61. Therefore, when seen in the first direction D1, at least a part of the heat dissipation member 25 overlaps each of the first lens frame 41 and the second lens frame 61. In addition, the first contact portion 26c is in contact with the lens frame 21. That is, the heat dissipation member 25 is in contact with the lens frame 21. The heat dissipation member 25 does not have to be in contact with both the first lens frame 41 and the second lens frame 61, and it is sufficient that the heat dissipation member 25 is in contact with at least one of the first lens frame 41 and the second lens frame 61.

The second heat dissipation member 27 is disposed closer to the second side (the −D2 side) in the second direction D2 than the objective lens unit 20 and the imaging unit 30. In this embodiment, a shape of the second heat dissipation member 27 has a shape that is plane-symmetric to a shape of the first heat dissipation member 26 with respect to a plane that passes through both the first axis J1 and the second axis J2. The second heat dissipation member 27 has a first fixing portion (not shown) and a second contact portion 27c.

The second fixing portion has a plate shape that extends in the optical axis direction. Although not shown, the second fixing portion is fixed by adhesive to a surface of the imaging element holding part 31 that faces the second side (the −D2 side) in the second direction D2. Thus, the second heat dissipation member 27 is in contact with the imaging element holding part 31. That is, the heat dissipation member 25 is in contact with the imaging element holding part 31. Therefore, the heat dissipation member 25 is in contact with the imaging unit 30.

The second contact portion 27c has a triangular prism shape that extends from an end portion of the second fixing portion on the object side (the +Z side) toward the object side. When seen in the optical axis direction, the second contact portion 27c has a substantially triangular shape that protrudes toward the first virtual line L1. In the second direction D2, a dimension of a portion of the second contact portion 27c in the first direction D1 that is farthest from the first virtual line L1 in the second direction D2 is larger than a dimension of a portion of the second contact portion 27c in the first direction D1 that is closest to the first virtual line L1. A part of the second contact portion 27c is disposed between the first lens frame 41 and the second lens frame 61, and is in contact with both the first lens frame 41 and the second lens frame 61. That is, at least a part of the heat dissipation member 25 is disposed between the first lens frame 41 and the second lens frame 61 and is in contact with both the first lens frame 41 and the second lens frame 61. Therefore, when seen in the first direction D1, at least a part of the heat dissipation member 25 overlaps each of the first lens frame 41 and the second lens frame 61. Furthermore, the second contact portion 27c is in contact with the lens frame 21. That is, the heat dissipation member 25 is in contact with the lens frame 21.

According to this embodiment, the endoscope 2 includes the lens frame 21 that holds the first objective optical system 42 and the second objective optical system 62 and extends in the optical axis direction, the imaging unit 30 having the imaging element 32, and the heat dissipation member 25 that is in contact with both the lens frame 21 and the imaging unit 30. Therefore, the heat generated in the imaging element 32 can be transferred to the objective lens unit 20 via the heat dissipation member 25. Thus, since it is possible to suppress the temperature of the imaging element 32 from becoming too high, the increase in dark current noise in the imaging element 32 can be suppressed. Therefore, since it is possible to suppress the image signals generated by the imaging element 32 from deteriorating, and the quality of the stereoscopic image of the subject can be improved.

Furthermore, in this embodiment, as described above, the heat generated in the imaging element 32 can be transferred to the lens frame 21 via the heat dissipation member 25. Therefore, since the heat generated in the imaging element 32 can be transferred to each of the lenses 42a constituting the first objective optical system 42 and each of the lenses 62a constituting the second objective optical system 62 via the heat dissipation member 25, it is possible to increase the temperature of each of the lenses 42a and 62a. Thus, even when the insertion part 6 of the endoscope 2 is inserted into the body, or the like, it is possible to suppress condensation from forming on the surfaces of the lenses 42a and 62a. As a result, it is possible to suppress deterioration in the quality of the optical image of the subject formed by the objective lens unit 20. Therefore, the quality of the stereoscopic image of the subject can be improved. Furthermore, compared to a case in which the optical unit 18 is additionally equipped with a heating member, such as a heater, for increasing the temperature of each of the lenses 42a and 62a, it is possible to suppress an increase in size of the optical unit 18 and it is also possible to suppress an increase in the number of parts of the optical unit 18. Therefore, it is possible to suppress an increase in size of the endoscope 2, and it is possible to suppress an increase in manufacturing cost of the endoscope 2.

According to this embodiment, the lens frame 21 includes the first lens frame 41 that holds the first objective optical system 42, and the second lens frame 61 that holds the second objective optical system 62, the imaging unit 30 has at least one imaging element 32, and at least a part of the heat dissipation member 25 is disposed between the first lens frame 41 and the second lens frame 61 and is in contact with at least one of the first lens frame 41 and the second lens frame 61. Therefore, since a part of the heat dissipation member 25 can be disposed in a space between the first lens frame 41 and the second lens frame 61, it is possible to suppress an increase in size of the optical unit 18 in the direction perpendicular to the optical axis direction. Therefore, it is possible to suppress the increase in size of the endoscope 2.

As described above, the endoscope 2 of this embodiment that forms a stereoscopic image of the subject on the basis of the optical image of the subject formed by each of the first objective optical system 42 and the second objective optical system 62 includes two imaging elements 32, that is, the first imaging element 32a and the second imaging element 32b, and the imaging elements 32a and 32b are disposed close to each other. Therefore, an amount of heat generated in the imaging element 32 increases, and the heat generated in each of the imaging elements 32a and 32b becomes difficult to dissipate. Therefore, the temperature of each of the imaging elements 32a and 32b is likely to become high. In contrast, in this embodiment, the heat dissipation member 25 is in contact with at least one of the first lens frame 41 and the second lens frame 61, and the imaging unit 30. Therefore, the heat generated in each of the imaging elements 32a and 32b can be transferred to the objective lens unit 20 via the heat dissipation member 25. Thus, since it is possible to suppress the temperature of each of the imaging elements 32a and 32b from becoming too high, the deterioration of the image signals generated in each of the imaging elements 32a and 32b can be suppressed. Therefore, the quality of the stereoscopic image of the subject can be improved.

As described above, in the endoscope 2 of this embodiment which forms a stereoscopic image, since the amount of heat generated in the imaging element 32 increases, the amount of heat that can be transferred to the objective lens unit 20 via the heat dissipation member 25 can be increased. Therefore, since condensation on the surfaces of each of the lenses 42a constituting the first objective optical system 42 and each of the lenses 62a constituting the second objective optical system 62 can be more effectively suppressed, it is possible to more effectively suppress a decrease in the quality of the optical image of the subject formed by the objective lens unit 20. Therefore, the quality of the stereoscopic image of the subject can be improved more suitably.

According to this embodiment, the heat dissipation member 25 is in contact with both the first lens frame 41 and the second lens frame 61. Therefore, since a contact area between the heat dissipation member 25 and the objective lens unit 20 can be increased, the amount of heat transferred to the objective lens unit 20 via the heat dissipation member 25 can be increased. Thus, since it is possible to suppress the temperature of each of the imaging elements 32a and 32b from becoming too high, deterioration of the image signals generated in each of the imaging elements 32a and 32b can be suppressed more effectively. Therefore, the quality of the stereoscopic image of the subject can be improved more suitably.

Furthermore, in this embodiment, as described above, since the heat dissipation member 25 is in contact with both the first lens frame 41 and the second lens frame 61, the amount of heat transferred to the objective lens unit 20 via the heat dissipation member 25 can be increased. Therefore, since condensation on the surfaces of each of the lenses 42a constituting the first objective optical system 42 and each of the lenses 62a constituting the second objective optical system 62 can be more effectively suppressed, it is possible to more effectively suppress the decrease in the quality of the optical image of the subject formed by the objective lens unit 20. Therefore, the quality of the stereoscopic image of the subject can be improved more suitably.

Furthermore, in this embodiment, the heat dissipation member 25 is disposed between the first lens frame 41 and the second lens frame 61 in the first direction D1, and is also in contact with both the first lens frame 41 and the second lens frame 61. Therefore, when the endoscope 2 is manufactured or repaired, even if a worker or the like accidentally grasps the two objective lens units 40 and 60 at once, it is possible to suppress each of the objective lens units 40 and 60 from being deformed in the first direction D1 due to the heat dissipation member 25. Therefore, damage to each of the objective lens units 40 and 60 during the manufacturing and repair of the endoscope 2 can be suppressed.

According to this embodiment, the first lens frame 41 and the second lens frame 61 are disposed with an interval therebetween in the first direction D1, and when seen in the first direction D1, at least a part of the heat dissipation member 25 overlaps each of the first lens frame 41 and the second lens frame 61. Therefore, compared to a case in which the heat dissipation member 25 is disposed further outward in the second direction D2 than each of the first lens frame 41 and the second lens frame 61, it is possible to suppress an increase in size of the optical unit 18 in the second direction D2. Therefore, it is possible to more effectively suppress an increase in size of the endoscope 2.

According to this embodiment, the imaging unit 30 includes the imaging element holding part 31 that holds the imaging element 32, the imaging element holding part 31 is made of a resin, and the heat dissipation member 25 is in contact with the imaging element holding part 31. Therefore, it is possible to suppress static electricity generated in the vicinity of the tip end portion 11 from being transmitted to the imaging element 32 via the heat dissipation member 25. Therefore, it is possible to suppress the imaging element 32 from being damaged by the static electricity, and also possible to suppress the image signal converted by the imaging element 32 from deteriorating. Therefore, the stability of the operation of the endoscope 2 can be improved.

According to this embodiment, the endoscope 2 includes the heat transfer members 38a and 38b that are disposed closer to the base end side (the −Z side) than the imaging element 32, and the heat transfer members 38a and 38b are in contact with the imaging unit 30. Therefore, as described above, since the heat generated in the imaging element 32 can be dissipated via the heat transfer members 38a and 38b, it is possible to more suitably suppress the temperature of the imaging element 32 from becoming too high. Therefore, since it is possible to more effectively suppress the image signals generated by the imaging element 32 from deteriorating, the quality of the stereoscopic image of the subject can be more effectively improved.

According to this embodiment, the heat dissipation member 25 has the contact portion 25c that is in contact with the lens frame 21, intersects the first axis J1 and the second axis J2 that are the central axes of the first lens frame 41 and the second lens frame 61, respectively, and also when a straight line that extends in the first direction D1 is defined as the first virtual line L1, in the second direction D2, a dimension of a portion of the contact portion 25c in the first direction D1 that is farthest from the first virtual line L1 in the second direction D2 is larger than a dimension of the portion of the contact portion 25c in the first direction D1 that is closest to the first virtual line L1. Therefore, a volume of the contact portion 25c that can be disposed in a space between the cylindrical first lens frame 41 and the cylindrical second lens frame 61 that are disposed side by side in the first direction D1 can be easily increased. Thus, since a heat capacity of the contact portion 25c can be easily increased, the amount of heat transferred to the objective lens unit 20 via the heat dissipation member 25 can be easily increased. Therefore, since it is possible to more effectively suppress the temperature of the imaging element 32 from becoming too high, the quality of the stereoscopic image of the subject can be more effectively improved. Furthermore, since the contact portion 25c can be easily disposed inward in the second direction D2, it is possible to suppress an increase in size of the optical unit 18 in the second direction D2.

Furthermore, in this embodiment, as described above, it is possible to further increase the amount of heat transferred to the objective lens unit 20 via the heat dissipation member 25. Therefore, condensation on the surfaces of each of the lenses 42a constituting the first objective optical system 42 and each of the lenses 62a constituting the second objective optical system 62 can be more effectively suppressed, and thus the quality of the stereoscopic image of the subject can be more effectively improved.

According to this embodiment, the heat dissipation member 25 includes the first heat dissipation member 26 disposed closer to a first side (the +D2 side) in the second direction D2 than the first virtual line L1, and the second heat dissipation member 27 disposed closer to a second side (the −D2 side) in the second direction D2 than the first virtual line L1. The first heat dissipation member 26 has the first contact portion 26c that is in contact with the lens frame 21, and the second heat dissipation member 27 has the second contact portion 27c that is in contact with the lens frame 21, and when seen in the optical axis direction, each of the first contact portion 26c and the second contact portion 27c has an approximately triangular shape that protrudes toward the first virtual line L1. Therefore, since the heat generated in the imaging element 32 can be transferred to the lens frame 21 via the first heat dissipation member 26 and the second heat dissipation member 27, thermal resistance between the imaging element 32 and the lens frame 21 can be reduced. Therefore, it is possible to further increase the amount of heat transferred to the objective lens unit 20 via the heat dissipation member 25. Therefore, since it is possible to more effectively suppress the temperature of the imaging element 32 from becoming too high, the quality of the stereoscopic image of the subject can be more effectively improved.

### First Modified Example of the First Embodiment

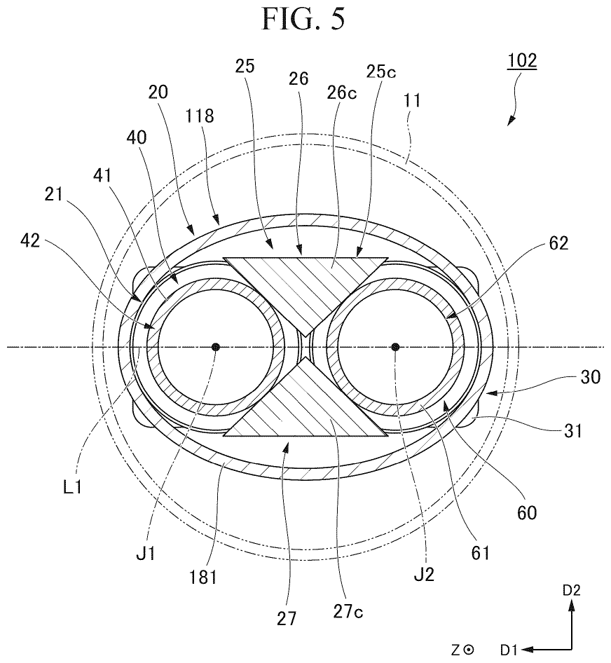

FIG. 5 is a cross-sectional view showing an optical unit 118 of this modified example. In the following description, the same components as those in the first embodiment described above are designated by the same reference numerals, and the description thereof will be omitted. As shown in FIG. 5, in this modified example, an endoscope 102 includes a holding frame 181.

The holding frame 181 has a cylindrical shape that extends in the optical axis direction. The holding frame 181 is open on both sides in the optical axis direction. When seen in the optical axis direction, the holding frame 181 has a generally elliptical shape of which a major axis extends in the first direction D1. When seen in the optical axis direction, the holding frame 181 may have another shape such as a rectangular shape. The holding frame 181 accommodates the first lens frame 41, the second lens frame 61, and the imaging unit 30. The holding frame 181 holds the first lens frame 41, the second lens frame 61, and the imaging unit 30. The holding frame 181 is provided with two objective lens unit holding holes (not shown). Each of the objective lens unit holding holes is a hole that passes through the holding frame 181 in the axial direction. The first objective lens unit 40 and the second objective lens unit 60 are passed through the different objective lens unit holding holes in the optical axis direction and are held by inner circumferential surfaces of the objective lens unit holding holes. Thus, the holding frame 181 holds the first lens frame 41 and the second lens frame 61.

In this modified example, the heat dissipation member 25 and the holding frame 181 are different members. The holding frame 181 is made of a resin. More specifically, the holding frame 181 is made of polyether ether ketone (PEEK). A thermal conductivity of the heat dissipation member 25 is greater than a thermal conductivity of the holding frame 181. Other configurations of the endoscope 102 of this modified example are similar to the other configurations of the endoscope 2 of the first embodiment described above.

According to this modified example, the endoscope 102 includes the holding frame 181 that accommodates the first lens frame 41, the second lens frame 61, and the imaging unit 30, and the heat dissipation member 25 and the holding frame 181 are different members. Therefore, a material constituting the holding frame 181 can be a resin material that is different from the metal material constituting the heat dissipation member 25. Therefore, the holding frame 181 having the objective lens unit holding holes can be molded by a molding method such as injection molding. Therefore, compared to a case in which the holding frame 181 is made of a metal material and each of the objective lens unit holding holes is formed by a processing method such as cutting, an increase in the number of manufacturing steps for the holding frame 181 can be suppressed. In addition, shape accuracy of each of the objective lens unit holding holes can be easily improved. Therefore, positional accuracy of the first objective lens unit 40 and the second objective lens unit 60 can be easily improved, and thus the quality of the optical image formed by the objective lens unit 20 can be easily improved.

When the holding frame 181 is made of a metal material, static electricity generated in the vicinity of the tip end portion 11 is easily transmitted to the imaging element 32 via the holding frame 181. In contrast thereto, in this modified example, since the holding frame 181 is made of a resin material, electrical resistance of the holding frame 181 can be increased. Therefore, it is possible to suppress the static electricity generated in the vicinity of the tip end portion 11 from being transmitted to the imaging element 32 via the holding frame 181. Therefore, it is possible to suppress the imaging element 32 being damaged by the static electricity, and also possible to suppress the image signal converted by the imaging element 32 from deteriorating. Therefore, the stability of the operation of the endoscope 102 can be improved.

According to this modified example, a thermal conductivity of the heat dissipation member 25 is greater than a thermal conductivity of the holding frame 181. Therefore, the amount of heat generated in the imaging element 32 that is transferred to the objective lens unit 20 via the heat dissipation member 25 can be increased relative to the amount of heat transferred to the holding frame 181. Thus, it is possible to suppress a decrease in the amount of heat transferred to the objective lens unit 20 via the heat dissipation member 25. Therefore, it is possible to more effectively suppress condensation on the surfaces of each of the lenses 42a constituting the first objective optical system 42 and each of the lenses 62a constituting the second objective optical system 62, and thus the quality of the stereoscopic image of the subject can be improved.

### Second Modified Example of the First Embodiment

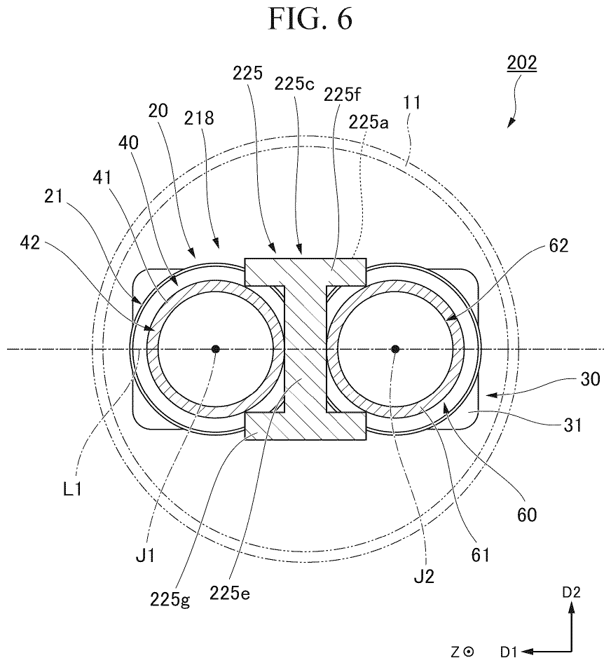

FIG. 6 is a cross-sectional view showing an optical unit 218 of this modified example. In the following description, the same components as those in the first embodiment described above are designated by the same reference numerals, and the description thereof will be omitted. As shown in FIG. 6, in this modified example, an endoscope 202 includes a heat dissipation member 225.

The heat dissipation member 225 of this modified example has a fixing portion 225a and a contact portion 225c. The configuration of the fixing portion 225a of this modified example is similar to the configuration of the first fixing portion 26a of the first embodiment described above.

Although not shown in the drawing, the contact portion 225c extends from an end of the fixing portion 225a on the object side (the +Z side) toward the object side. When seen in the optical axis direction, the contact portion 225c has an I-shape. The contact portion 225c includes a first portion 225e and second portions 225f and 225g. The first portion 225e has a plate shape that extends in the second direction D2. A plate surface of the first portion 225e faces the first direction D1. The first portion 225e passes through a space between the first lens frame 41 and the second lens frame 61 in the second direction D2. Therefore, at least a part of the heat dissipation member 225 overlaps each of the first lens frame 41 and the second lens frame 61 when seen in the first direction D1. In this modified example, the first portion 225e is in contact with the first lens frame 41 and the second lens frame 61. That is, at least a part of the heat dissipation member 225 is disposed between the first lens frame 41 and the second lens frame 61, and is in contact with both the first lens frame 41 and the second lens frame 61. Furthermore, the contact portion 225c is in contact with the lens frame 21. That is, the heat dissipation member 225 is in contact with the lens frame 21. An end portion of the first portion 225e on a first side (the +D2 side) in the second direction D2 is located closer to a first side in the second direction D2 than the first virtual line L1. An end portion of the first portion 225e on the second side (the −D2 side) in the second direction D2 is located closer to the second side in the second direction D2 than the first virtual line L1.

In this modified example, the heat dissipation member 225 has two second portions 225f and 225g. One of the second portions 225f extends in the first direction D1 from an end portion of the first portion 225e on a first side (the +D2 side) in the second direction D2. In this modified example, one second portion 225f extends to both sides in the first direction D1 from the first portion 225e. The other second portion 225g extends in the first direction D1 from an end portion of the first portion 225e on a second side (the −D2 side) in the second direction D2. In this modified example, the other second portion 225g extends to both sides in the first direction D1 from the first portion 225e. As a result, the second portions 225f and 225g extend in the first direction D1 from both ends of the first portion 225e in the second direction D2. In this modified example, the second portions 225f and 225g are in contact with the first lens frame 41 and the second lens frame 61. Thus, the contact portion 225c is in contact with the lens frame 21. That is, the heat dissipation member 225 is in contact with the lens frame 21. The heat dissipation member 225 does not have to be in contact with both the first lens frame 41 and the second lens frame 61, and only needs to be in contact with at least one of the first lens frame 41 and the second lens frame 61.

The dimension of each of the second portions 225f and 225g in the first direction D1 is greater than the dimension of the first portion 225e in the first direction D1. Therefore, the dimension of a portion of the contact portion 225c in the first direction D1 that is farthest from the first virtual line L1 in the second direction D2 is larger than a dimension of a portion of the contact portion 225c in the first direction D1 that is closest to the first virtual line L1, that is, in this modified example, a portion of the first portion 225e through which the first virtual line L1 passes. Other configurations of the heat dissipation member 225 of this modified example are similar to other configurations of the heat dissipation member 25 of the first embodiment described above.

According to this modified example, the contact portion 225c has the first portion 225e that extends in the second direction D2, and the second portions 225f and 225g that extend in the first direction D1 from both ends of the first portion 225e in the second direction D2. Therefore, it is possible to increase rigidity of the contact portion 225c in the first direction D1 and the second direction D2. Thus, the contact portion 225c can be in stable contact with both the first lens frame 41 and the second lens frame 61. Thus, it is possible to suppress an increase in thermal resistance between the heat dissipation member 225 and the first lens frame 41 and between the heat dissipation member 225 and the second lens frame 61. Therefore, it is possible to stabilize the amount of heat transferred from the imaging unit 30 to the objective lens unit 20 via the heat dissipation member 225. Therefore, since it is possible to suppress the temperature of the imaging element 32 from becoming too high, the quality of the stereoscopic image of the subject can be improved.

Furthermore, in this modified example, as described above, the amount of heat transferred to the objective lens unit 20 via the heat dissipation member 225 can be stabilized. Therefore, since condensation on the surfaces of each of the lenses 42a constituting the first objective optical system 42 and each of the lenses 62a constituting the second objective optical system 62 can be reliably suppressed, the quality of the stereoscopic image of the subject can be more suitably improved.

### Third Modified Example of the First Embodiment

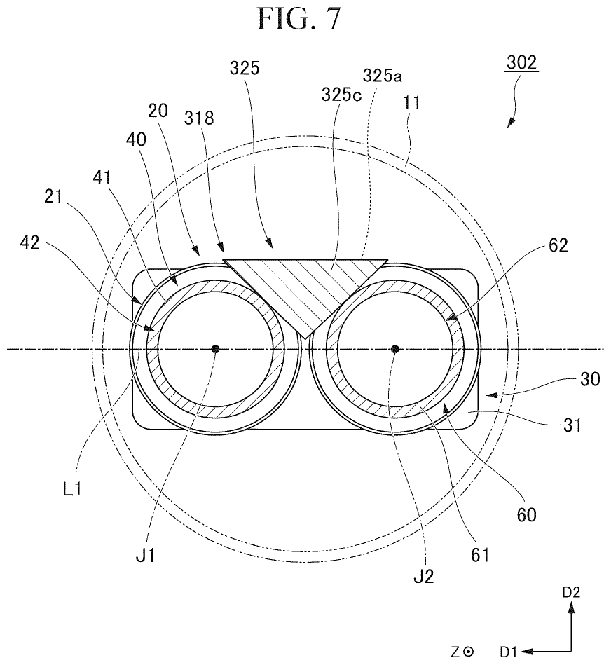

FIG. 7 is a cross-sectional view showing an optical unit 318 of this modified example. In the following description, the same components as those in the first embodiment described above are designated by the same reference numerals, and the description thereof will be omitted. As shown in FIG. 7, in this modified example, an endoscope 302 includes a heat dissipation member 325.

The heat dissipation member 325 of this modified example has a fixing portion 325a and a contact portion 325c. The configuration of the heat dissipation member 325 of this modified example is similar to the configuration of the first heat dissipation member 26 of the first embodiment described above. The configurations of the fixing portion 325a and the contact portion 325c of this modified example are similar to the configurations of the first fixing portion 26a and the first contact portion 26c of the first embodiment described above.

When seen in the optical axis direction, the contact portion 325c has a substantially triangular shape that protrudes toward the first virtual line L1. In the following description, the substantially triangular shape includes a shape in which at least one of three corners of the triangle is linear or curved. A dimension of a portion of the contact portion 325c in the first direction D1 that is farthest from the first virtual line L1 in the second direction D2 is larger than a dimension of a portion of the contact portion 325c in the first direction D1 that is closest to the first virtual line L1. A part of the contact portion 325c is disposed between the first lens frame 41 and the second lens frame 61, and is in contact with both the first lens frame 41 and the second lens frame 61. That is, at least a part of the heat dissipation member 325 is disposed between the first lens frame 41 and the second lens frame 61, and is in contact with both the first lens frame 41 and the second lens frame 61. Therefore, when seen in the first direction D1, at least a part of the heat dissipation member 325 overlaps each of the first lens frame 41 and the second lens frame 61. In addition, the contact portion 325c is in contact with the lens frame 21. That is, the heat dissipation member 325 is in contact with the lens frame 21. Other configurations of the heat dissipation member 325 of this modified example are similar to other configurations of the heat dissipation member 25 of the first embodiment described above.

According to this modified example, when seen in the optical axis direction, the contact portion 325c has a substantially triangular shape that protrudes toward the first virtual line L1. Therefore, since the shape of the contact portion 325c can be simplified, an increase in the number of steps required for forming the contact portion 325c by cutting can be suppressed. Therefore, an increase in the number of steps and the manufacturing cost of the heat dissipation member 325 can be suppressed.

According to this modified example, the contact portion 325c is in contact with the lens frame 21. Therefore, the heat generated in the imaging unit 30 can be transferred to the objective lens unit 20 via the heat dissipation member 325. Therefore, since it is possible to suppress the temperature of the imaging element 32 from becoming too high, the quality of the stereoscopic image of the subject can be improved.

Furthermore, in this modified example, as described above, the heat generated in the imaging unit 30 can be transferred to the objective lens unit 20 via the heat dissipation member 325. Therefore, since condensation on the surfaces of each of the lenses 42a constituting the first objective optical system 42 and each of the lenses 62a constituting the second objective optical system 62 can be reliably suppressed, the quality of the stereoscopic image of the subject can be more suitably improved.

### Fourth Modified Example of the First Embodiment

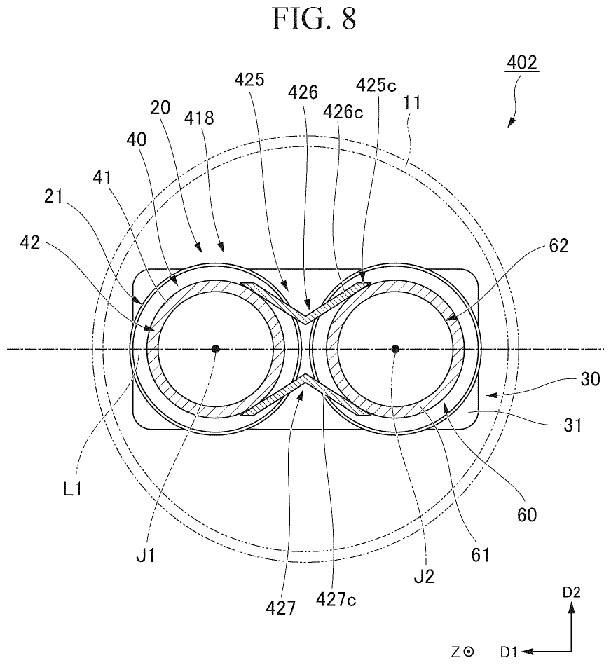

FIG. 8 is a cross-sectional view showing an optical unit 418 of this modified example. In the following description, the same components as those in the first embodiment described above are designated by the same reference numerals, and the description thereof will be omitted. As shown in FIG. 8, in this modified example, an endoscope 402 includes a heat dissipation member 425.

The heat dissipation member 425 has a contact portion 425c that is in contact with the lens frame 21. The contact portion 425c includes a first contact portion 426c and a second contact portion 427c which will be described below. In this modified example, the endoscope 402 includes two heat dissipation members 425. The heat dissipation member 425 includes a first heat dissipation member 426 and a second heat dissipation member 427. The first heat dissipation member 426 is disposed closer to first side (the +D2 side) in the second direction D2 than the first virtual line L1. The second heat dissipation member 427 is disposed closer to the second side (the −D2 side) in the second direction D2 than the first virtual line L1.

The first heat dissipation member 426 has a first fixing portion (not shown) and a first contact portion 426c. The configuration of the first fixing portion of this modified example is similar to the configuration of the first fixing portion 26a of the first embodiment described above.

When seen in the optical axis direction, the first contact portion 426c has a V-shape that widens in the first direction D1 as it moves away from the first virtual line L1 in the second direction D2. In other words, when seen in the optical axis direction, the contact portion 425c has a V-shape that widens in the first direction D1 as it moves away from the first virtual line L1 in the second direction D2. A dimension of a portion of the first contact portion 426c in the first direction D1 that is farthest from the first virtual line L1 in the second direction D2 is larger than a dimension of a portion of the first contact portion 426c in the first direction D1 that is closest to the first virtual line L1. A part of the first contact portion 426c is disposed between the first lens frame 41 and the second lens frame 61, and is in contact with both the first lens frame 41 and the second lens frame 61. That is, at least a part of the heat dissipation member 425 is disposed between the first lens frame 41 and the second lens frame 61, and is in contact with both the first lens frame 41 and the second lens frame 61. Therefore, when seen in the first direction D1, at least a part of the heat dissipation member 425 overlaps each of the first lens frame 41 and the second lens frame 61. In addition, the first contact portion 426c is in contact with the lens frame 21. That is, the heat dissipation member 425 is in contact with the lens frame 21. Other configurations of the first heat dissipation member 426 are similar to other configurations of the first heat dissipation member 26 of the first embodiment described above.

The second heat dissipation member 427 has a first fixing portion (not shown) and a second contact portion 427c. The configuration of the second fixing portion of this modified example is similar to the configuration of the second fixing portion of the first embodiment described above.

When seen in the optical axis direction, the second contact portion 427c has a V-shape that widens in the first direction D1 as it moves away from the first virtual line L1 in the second direction D2. A dimension of a portion of the second contact portion 427c in the first direction D1 that is farthest from the first virtual line L1 in the second direction D2 is larger than a dimension of a portion of the second contact portion 427c in the first direction D1 that is closest to the first virtual line L1. A part of the second contact portion 427c is disposed between the first lens frame 41 and the second lens frame 61, and is in contact with both the first lens frame 41 and the second lens frame 61. That is, at least a part of the heat dissipation member 425 is disposed between the first lens frame 41 and the second lens frame 61, and is in contact with both the first lens frame 41 and the second lens frame 61. Therefore, when seen in the first direction D1, at least a part of the heat dissipation member 425 overlaps each of the first lens frame 41 and the second lens frame 61. In addition, the second contact portion 427c is in contact with the lens frame 21. That is, the heat dissipation member 425 is in contact with the lens frame 21. Other configurations of the second heat dissipation member 427 are similar to other configurations of the second heat dissipation member 27 of the first embodiment described above.

According to this modified example, when seen in the optical axis direction, the contact portion 425c has a V-shape that widens in the first direction D1 as it moves away from the first virtual line L1 in the second direction D2. Therefore, the contact portion 425c can be formed by bending a metal sheet. Therefore, since the contact portion 425c can be easily formed, it is possible to suppress an increase in the number of manufacturing steps for the contact portion 425c. Therefore, it is possible to suppress an increase in the number of manufacturing steps and manufacturing costs for the heat dissipation member 425.

Furthermore, in this modified example, the contact portion 425c is in contact with the lens frame 21. Therefore, the heat generated in the imaging unit 30 can be transferred to the objective lens unit 20 via the heat dissipation member 425. Therefore, since it is possible to suppress the temperature of the imaging element 32 from becoming too high, the quality of the stereoscopic image of the subject can be improved.

Furthermore, in this modified example, as described above, the heat generated in the imaging unit 30 can be transferred to the objective lens unit 20 via the heat dissipation member 425. Therefore, since condensation on the surfaces of each of the lenses 42a constituting the first objective optical system 42 and each of the lenses 62a constituting the second objective optical system 62 can be reliably suppressed, the quality of the stereoscopic image of the subject can be more suitably improved.

### Second Embodiment

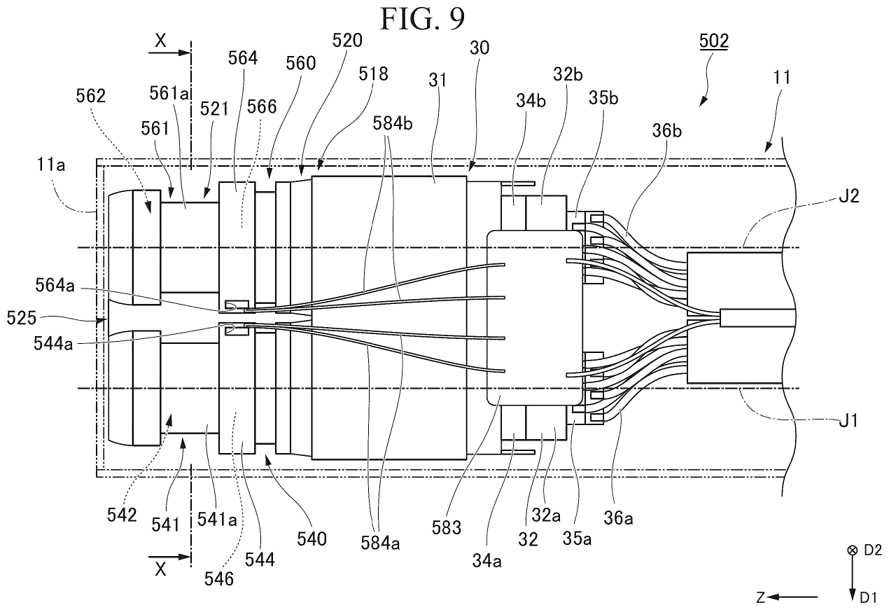

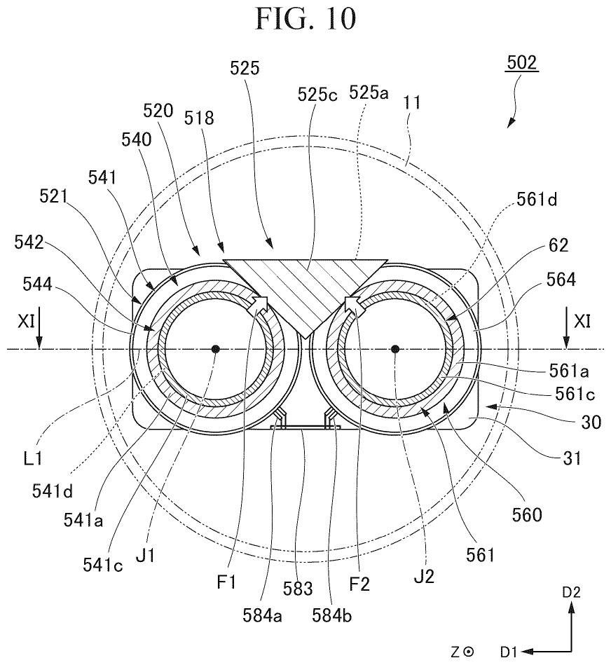

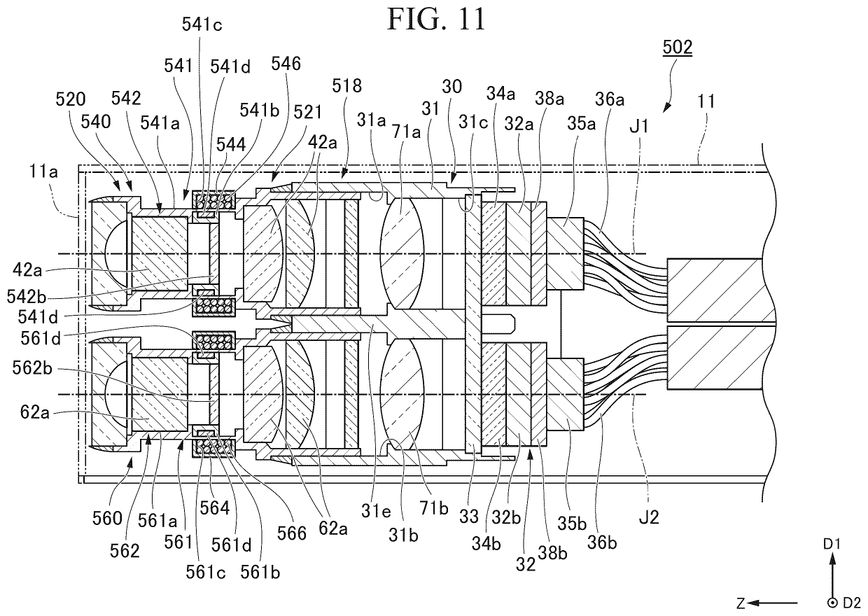

FIG. 9 is an external view of an optical unit 518 of this embodiment when seen from the second side (the −D2 side) in the second direction D2. FIG. 10 is a cross-sectional view showing the optical unit 518 of this embodiment and taken along line X-X in FIG. 9. FIG. 11 is a cross-sectional view showing the optical unit 518 of this embodiment and taken along line XI-XI in FIG. 10. In the following description, the same components as those in the third modified example of the first embodiment described above are designated by the same reference numerals, and the description thereof will be omitted. As shown in FIG. 9, the optical unit 518 includes an objective lens unit 520, a heat dissipation member 525, the imaging unit 30, and a board 583.

The objective lens unit 520 includes a first objective lens unit 540 and a second objective lens unit 560. As shown in FIG. 10, the first objective lens unit 540 and the second objective lens unit 560 are disposed to be spaced apart from each other in the first direction D1. The objective lens unit 520 includes a lens frame 521. The lens frame 521 includes a first lens frame 541 and a second lens frame 561.

As shown in FIG. 11, the first objective lens unit 540 includes a first lens frame 541 and a first objective optical system 542. The first lens frame 541 has a substantially cylindrical shape that extends in the optical axis direction with the first axis J1 as a center thereof. The first lens frame 541 has a first holding portion 541a, a first moving frame 541b, and a first coil accommodation portion 544.

The first holding portion 541a has a substantially cylindrical shape that extends in the optical axis direction with the first axis J1 as a center thereof. The first holding portion 541a is open on both sides in the optical axis direction. A portion of the first holding portion 541a on the base end side (the −Z side) is inserted into the inside of the first holding hole 31a of the imaging element holding part 31 and is fixed to an inner circumferential surface of the first holding hole 31a. Thus, the imaging element holding part 31 holds the first objective lens unit 540.

The first moving frame 541b has a substantially cylindrical shape that protrudes in the optical axis direction with the first axis J1 as a center thereof. The first moving frame 541b is open on both sides in the optical axis direction. The first moving frame 541b is disposed inside the first holding portion 541a. A part of an outer circumferential surface of the first moving frame 541b is in contact with an inner circumferential surface of the first holding portion 541a. The first moving frame 541b is held by the first holding portion 541a so as to be movable in the optical axis direction. Thus, the first moving frame 541b is movable in the optical axis direction. A first magnet mounting hole 541c is provided in the first moving frame 541b. The first magnet mounting hole 541c is a hole recessed radially inward from the outer circumferential surface of the first moving frame 541b with the first axis J1 as a center thereof. The first magnet mounting hole 541c is provided around the entire circumferential direction.

A magnet 541d is disposed inside the first magnet mounting hole 541c. In this embodiment, a plurality of magnets 541d are disposed inside the first magnet mounting hole 541c. In this embodiment, the magnets 541d are disposed in a circumferential direction. A shape of each of the magnets 541d is not particularly limited, and may be a flat plate that extends in the optical axis direction or an arc that extends in the circumferential direction. In addition, the number of magnets 541d is not particularly limited, and is preferably 2 or more and 4 or less. The number of magnets 541d may be 1. Each of the magnets 541d is mounted on an inner surface of the first magnet mounting hole 541c. That is, the magnet 541d is mounted in the first moving frame 541b. The first moving frame 541b also holds a moving lens 542b. That is, the first 5 moving frame 541b holds at least one lens. The moving lens 542b is movable in the optical axis direction together with the first moving frame 541b.

The first coil accommodation portion 544 has a substantially cylindrical shape that protrudes in the optical axis direction with the first axis J1 as a center thereof. The first coil accommodation portion 544 is fixed to an outer circumferential surface of the first holding portion 541a by adhesive. As shown in FIG. 9, an opening 544a is provided in a portion of the first coil accommodation portion 544 on the second side (the −D2 side) in the second direction D2. The opening 544a connects the inside and outside of the first coil accommodation portion 544. As shown in FIG. 11, a coil 546 is disposed inside the first coil accommodation portion 544. In this embodiment, the first coil accommodation portion 544 is made of a magnetic material such as iron or steel. The first coil accommodation portion 544 may be used as a yoke that forms a magnetic circuit between the first coil accommodation portion 544 and the magnet 541d.

The coil 546 is wound around the outer circumferential surface of the first holding portion 541a. More specifically, the coil 546 is wound around the outer circumferential surface of the first holding portion 541a in the circumferential direction of the first axis J1. Thus, the coil 546 is mounted on the first holding portion 541a.

The first objective optical system 542 forms an optical image of the subject. An optical axis of the first objective optical system 542 overlaps the first axis J1 when seen in the optical axis direction. The optical axis of the first objective optical system 542 does not have to overlap the first axis J1 when seen in the optical axis direction. The first objective optical system 542 is disposed inside the first lens frame 541. The first objective optical system 542 is configured of the plurality of lenses 42a and the moving lens 542b disposed in the optical axis direction. In this embodiment, the configuration of the plurality of lenses 42a is similar to that of the plurality of lenses 42a of the first embodiment. The plurality of lenses 42a are held by the inner circumferential surface of the first holding portion 541a. As described above, the moving lens 542b is held by the first moving frame 541b, and is movable in the optical axis direction together with the first moving frame 541b. A focal position of the optical image of the subject formed by the first objective optical system 542 can be adjusted by adjusting a position of the moving lens 542b in the optical axis direction. Other configurations of the first objective optical system 542 are similar to other configurations of the first objective optical system 42 of the first embodiment described above.

The second objective lens unit 560 includes a second lens frame 561 and a second objective optical system 562. The second lens frame 561 has a substantially cylindrical shape that extends in the optical axis direction with the second axis J2 as a center thereof. The shape of the second lens frame 561 is substantially the same as the shape of the first lens frame 541. As shown in FIG. 10, the second lens frame 561 is disposed closer to the second side (the −D1 side) in the first direction D1 than the first lens frame 541. The first lens frame 541 and the second lens frame 561 are disposed with an interval therebetween in the first direction D1. As shown in FIG. 11, the second lens frame 561 has a second holding portion 561a, a second moving frame 561b, and a second coil accommodation portion 564.

The second holding portion 561a has a substantially cylindrical shape that extends in the optical axis direction with the second axis J2 as a center thereof. The second holding portion 561a is open on both sides in the optical axis direction. A portion of the second holding portion 561a on the base end side (the −Z side) is inserted into the second holding hole 31b of the imaging element holding part 31 and fixed to an inner circumferential surface of the second holding hole 31b. Thus, the imaging element holding part 31 holds the second objective lens unit 560.

The second moving frame 561b has a substantially cylindrical shape that protrudes in the optical axis direction with the second axis J2 as a center thereof. The second moving frame 561b is open on both sides in the optical axis direction. The second moving frame 561b is disposed inside the second holding portion 561a. The second moving frame 561b is held by the second holding portion 561a so as to be movable in the optical axis direction. Thus, the second moving frame 561b is movable in the optical axis direction. A second magnet mounting hole 561c is provided in the second moving frame 561b. The second magnet mounting hole 561c is a hole recessed radially inward from an outer circumferential surface of the second moving frame 561b with the second axis J2 as a center thereof. The second magnet mounting hole 561c is provided around the entire circumferential direction.

A magnet 561d is disposed inside the second magnet mounting hole 561c. In this embodiment, a plurality of magnets 561d are disposed inside the second magnet mounting hole 561c. In this embodiment, each of the magnets 561d is disposed in the circumferential direction. The shape of each of the magnets 561d is not particularly limited, and may be a flat plate that extends in the optical axis direction or an arc that extends in the circumferential direction. The number of magnets 561d is not particularly limited, and is preferably two or more and four or less. The number of magnets 561d may be one. Each of the magnets 561d is mounted on an inner surface of the second magnet mounting hole 561c. That is, the magnets 561d are mounted in the second moving frame 561b. The second moving frame 561b also holds a moving lens 562b. That is, the second moving frame 561b holds at least one lens. The moving lens 562b is movable in the optical axis direction together with the second moving frame 561b.

The second coil accommodation portion 564 has a substantially cylindrical shape that protrudes in the optical axis direction with the second axis J2 as a center thereof. The second coil accommodation portion 564 is fixed to the outer circumferential surface of the second holding portion 561a by adhesive. As shown in FIG. 9, an opening 564a is provided in a portion of the second coil accommodation portion 564 on the second side (the −D2 side) in the second direction D2. The opening 564a connects the inside and outside of the second coil accommodation portion 564. As shown in FIG. 11, a coil 566 is disposed inside the second coil accommodation portion 564. In this embodiment, the second coil accommodation portion 564 is made of a magnetic material such as iron or steel. The second coil accommodation portion 564 may be used as a yoke that forms a magnetic circuit between the second coil accommodation portion 564 and the magnet 561d.

The coil 566 is wound around an outer circumferential surface of the second holding portion 561a. More specifically, the coil 566 is wound around the outer circumferential surface of the second holding portion 561a in the circumferential direction of the second axis J2. The coil 566 is mounted on the second holding portion 561a.

The second objective optical system 562 forms an optical image of the subject. An optical axis of the second objective optical system 562 overlaps the second axis J2 when seen in the optical axis direction. The optical axis of the second objective optical system 562 does not have to overlap the second axis J2 when seen in the optical axis direction. The second objective optical system 562 is disposed inside the second lens frame 561. The second objective optical system 562 is configured of a plurality of lenses 62a and a moving lens 562b disposed in the optical axis direction. In this embodiment, the configuration of the plurality of lenses 62a is similar to that of the plurality of lenses 62a in the first embodiment. The plurality of lenses 62a are held by the inner circumferential surface of the second holding portion 561a. As described above, the moving lens 562b is held by the second moving frame 561b, and is movable in the optical axis direction together with the second moving frame 561b. A focal position of an optical image of the subject formed by the second objective optical system 562 can be adjusted by adjusting a position of the moving lens 562b in the optical axis direction. Other configurations of the second objective optical system 562 are similar to those of the second objective optical system 62 of the first embodiment described above.

The board 583 shown in FIG. 9 controls a current supplied to the coils 546 and 566. In this embodiment, the board 583 is, for example, a flexible printed circuit board. An inverter circuit (not shown) that generates a current to be supplied to the coils 546 and 566 is mounted on the board 583. The board 583 is fixed to a surface of the imaging element holding part 31 that faces the second side (the −D2 side) in the second direction D2. The board 583 is electrically connected to the imaging cable bundles 36a and 36b. Thus, the board 583 and the processor 3 are communicatively connected. Furthermore, the board 583 is electrically connected to the coil 546 via a connection line 584a, and is electrically connected to the coil 566 via a connection line 584b.

In the endoscope 502 of this embodiment, a user can adjust the focal position of the optical image of the subject by operating the switch 16 (refer to FIG. 1) of the operation part 7. When the user or the like operates the switch 16, a movement signal which is a signal to move the first moving frame 541b and the second moving frame 561b in the optical axis direction is transmitted from the processor 3 to the board 583. On the basis of the movement signal, the board 583 adjusts a magnitude of the current supplied to the coils 546 and 566 and a direction of the current flow. When a current flows through the coil 546, the first moving frame 541b moves in the optical axis direction due to a magnetic force between an electromagnet formed by the coil 546 and the magnet 541d held by the first moving frame 541b. Similarly, when a current flows through the coil 566, the second moving frame 561b moves in the optical axis direction due to a magnetic force between an electromagnet formed by the coil 566 and the magnet 561d held by the second moving frame 561b. That is, each of the first moving frame 541b and the second moving frame 561b moves in the optical axis direction by passing a current through the coils 546 and 566. Thus, positions of the moving lenses 542b and 562b in the optical axis direction can be adjusted by passing a current through the coils 546 and 566, and thus the focal positions of the optical images of the subject formed by the first objective optical system 542 and the second objective optical system 562 can be adjusted.

The heat dissipation member 525 transfers heat generated in the imaging element 32 to the lens frame 521. The heat dissipation member 525 of this embodiment is made of a magnetic material. Examples of materials that can be used to form the heat dissipation member 525 include iron, silicon steel, permalloy, and the like. Other configurations of the heat dissipation member 525 are similar to those of the heat dissipation member 325 of the third modified example of the first embodiment described above. As shown in FIG. 10, the heat dissipation member 525 is disposed only on a first side (the +D2 side) in the second direction D2 with respect to the first virtual line L1. The heat dissipation member 525 has a fixing portion 525a and a contact portion 525c. Although not shown in the drawing, the fixing portion 525a is fixed by adhesion to a surface of the imaging element holding part 31 that faces a first side in the second direction D2. Thus, the heat dissipation member 525 is in contact with the imaging unit 30. Other configurations of the fixing portion 525a are similar to other configurations of the fixing portion 325a described above.

When seen in the optical axis direction, the contact portion 525c has a substantially triangular shape that protrudes toward the first virtual line L1. A part of the contact portion 525c is disposed between the first lens frame 541 and the second lens frame 561, and is in contact with both the first lens frame 541 and the second lens frame 561. Thus, the heat dissipation member 525 is in contact with the lens frame 521. As described above, since the contact portion 525c is made of a magnetic material, a magnetic force F1 is generated between the contact portion 525c and the magnet 541d, and a magnetic force F2 is generated between the contact portion 525c and the magnet 561d. Thus, since the magnetic force F1 directed toward the contact portion 525c is applied to the first moving frame 541b, the position of the first moving frame 541b relative to the first holding portion 541a is determined in the radial and circumferential directions with the first axis J1 as a center thereof. Furthermore, since the magnetic force F2 directed toward the contact portion 525c is applied to the second moving frame 561b, the position of the second moving frame 561b relative to the second holding portion 561a is determined in the radial and circumferential directions with the second axis J2 as a center thereof. Thus, it is possible to stabilize the positions of the moving lenses 542b and 562b with respect to the lens frame 521. Other configurations of the contact portion 525c are similar to other configurations of the contact portion 325c described above.

According to this embodiment, each of the first moving frame 541b and the second moving frame 561b holds at least one lens, that is, each of the moving lenses 542b and 562b, the magnets 541d and 561d are mounted in the first moving frame 541b and the second moving frame 561b, respectively, the coils 546 and 566 are mounted in the first holding portion 541a and the second holding portion 561a, respectively, and the first moving frame 541b and the second moving frame 561b move in the optical axis direction by passing a current through the coils 546 and 566. Therefore, the positions of the moving lenses 542b and 562b in the optical axis direction can be adjusted as described above by adjusting a current flowing through the coils 546 and 566. Thus, the focal positions of the optical images of the subject formed by each of the first objective optical system 542 and the second objective optical system 562 can be adjusted. Therefore, since the endoscope 502 of this embodiment can form a clearer stereoscopic image of the subject, the quality of the stereoscopic image of the subject can be more suitably improved.

According to this embodiment, the heat dissipation member 525 is made of a magnetic material. Therefore, as described above, the magnetic force F1 is generated between the contact portion 525c and the magnet 541d, and the magnetic force F2 is generated between the contact portion 525c and the magnet 561d. Thus, even when there is play between the first holding portion 541a and the first moving frame 541b due to dimensional tolerances of the first holding portion 541a and the first moving frame 541b, the position of the first moving frame 541b with respect to the first holding portion 541a can be stabilized. Therefore, the position of the moving lens 542b with respect to the first holding portion 541a can be stabilized. Similarly, even when there is play between the second holding portion 561a and the second moving frame 561b, the position of the second moving frame 561b with respect to the second holding portion 561a can be stabilized. Therefore, the position of the moving lens 562b with respect to the second holding portion 561a can be stabilized. Thus, since the optical images of the subject formed by the first objective optical system 542 and the second objective optical system 562 can be stabilized, the quality of the stereoscopic image can be improved more suitably.

According to this embodiment, the heat dissipation member 525 is disposed only on a first side (the +D2 side) in the second direction D2 with respect to the first virtual line L1. Therefore, the direction of the magnetic force applied to the magnets 541d and 561d can be stabilized compared to a case in which the heat dissipation member 525 is disposed on both a first side in the second direction D2 and a second side (the −D2 side) in the second direction D2 with respect to the first virtual line L1. Thus, it is possible to more suitably stabilize the position of the moving lens 542b with respect to the first holding portion 541a and the position of the moving lens 562b with respect to the second holding portion 561a. Therefore, since the optical images of the subject formed by the first objective optical system 542 and the second objective optical system 562 can be more suitably stabilized, the quality of the stereoscopic image can be more suitably improved.

Furthermore, in this embodiment, the heat dissipation member 525 is in contact with the imaging unit 30 and the lens frame 521. Therefore, the heat generated in the imaging unit 30 can be transferred to the objective lens unit 520 via the heat dissipation member 525. Therefore, since it is possible to suppress the temperature of the imaging element 32 from becoming too high, and the quality of the stereoscopic image of the subject can be improved.

Furthermore, in this embodiment, as described above, the heat generated in the imaging unit 30 can be transferred to the objective lens unit 520 via the heat dissipation member 525. Therefore, since condensation on the surfaces of each of the lenses constituting the first objective optical system 542 and each of the lenses constituting the second objective optical system 562 can be reliably suppressed, the quality of the stereoscopic image of the subject can be more suitably improved.

### Third Embodiment

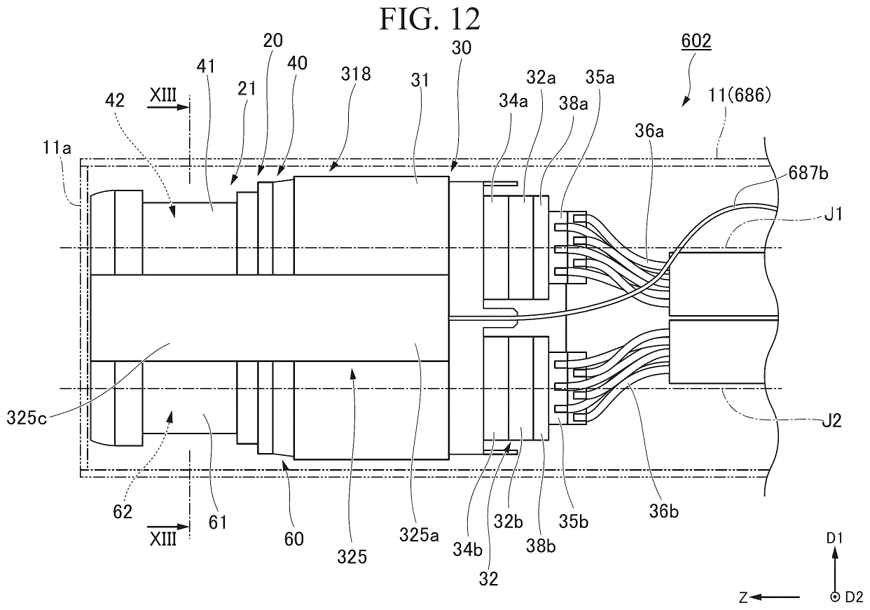

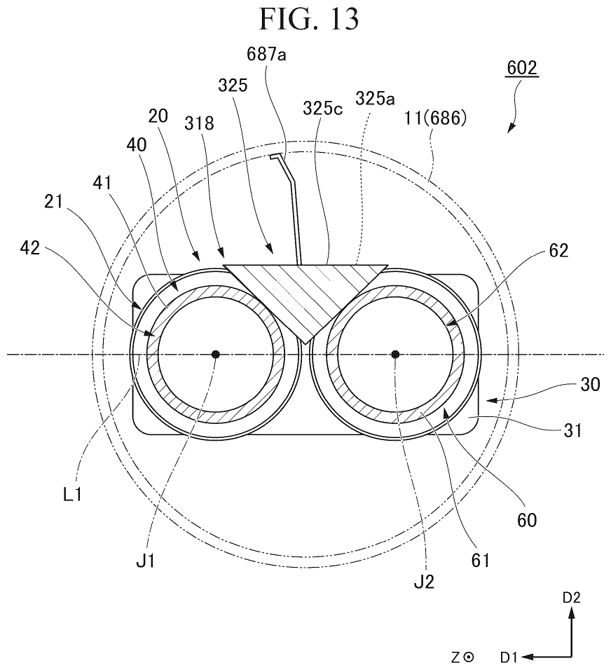

FIG. 12 is an external view of an optical unit 318 of this embodiment when seen from a first side (the +D2 side) in the second direction D2. FIG. 13 is a cross-sectional view showing the optical unit 318 of this embodiment and taken along line XIII-XIII in FIG. 12. In the following description, the same components as those in the third modified example of the first embodiment described above are designated by the same reference numerals, and a description thereof will be omitted. As shown in FIGS. 12 and 13, an endoscope 602 of this embodiment includes the optical unit 318, the heat dissipation member 325, the imaging unit 30, a heat-conducted member 686, and heat transfer members 687a and 687b.

The heat generated in the imaging element 32 is transferred to the heat-conducted member 686. In this embodiment, the heat-conducted member 686 includes metallic members such as the tip end portion 11 and the rigid tube portion 13 (refer to FIG. 1). The heat-conducted member 686 may be a member other than the tip end portion 11 and the rigid tube portion 13.

As shown in FIG. 13, the heat transfer member 687a is a leaf spring that extends from the heat dissipation member 325 toward the tip end portion 11. The heat transfer member 687a is made of a metal. The heat transfer member 687a is in contact with both the heat dissipation member 325 and the tip end portion 11. Thus, the heat transfer member 687a connects the heat dissipation member 325 and the heat-conducted member 686. Some of the heat generated in the imaging element 32 is transferred to the tip end portion 11 via the heat dissipation member 325 and the heat transfer member 687a. The heat transferred to the tip end portion 11 is dissipated to the outside of the endoscope 602. The configuration of the heat transfer member 687a is not limited to this embodiment, and as long as it is possible to transfer the heat of the heat dissipation member 325 to the tip end portion 11, it may be made of other materials and have other shapes, such as a sheet-shaped member made of carbon.

As shown in FIG. 12, the heat transfer member 687b is a cable that extends from the heat dissipation member 325 toward the base end side (the −Z side). Although not shown, the heat transfer member 687b is in contact with both the heat dissipation member 325 and the rigid tube portion 13. Thus, the heat transfer member 687b connects the heat dissipation member 325 and the heat-conducted member 686. Some of the heat generated in the imaging element 32 is transferred to the rigid tube portion 13 via the heat dissipation member 325 and the heat transfer member 687b. The heat transferred to the rigid tube portion 13 is dissipated to the outside of the endoscope 602. The configuration of the heat transfer member 687b is not limited to this embodiment, and is not limited to this embodiment as long as it is possible to transfer the heat of the heat dissipation member 325 to the rigid tube portion 13. In addition, the heat transfer member 687b may connect the heat dissipation member 325 and the tip end portion 11.

According to this embodiment, the endoscope 602 includes the heat-conducted member 686, and the heat transfer members 687a and 687b that connect the heat dissipation member 325 and the heat-conducted member 686. Therefore, the heat generated in the imaging element 32 can be transferred to the heat-conducted member 686 in addition to the objective lens unit 20. Therefore, even when the imaging element 32 is used continuously, or when the imaging element 32 generates a large amount of heat, it is possible to more effectively suppress the temperature of the imaging element 32 from becoming too high. Therefore, the quality of the stereoscopic image of the subject can be improved more suitably.

### Fourth Embodiment

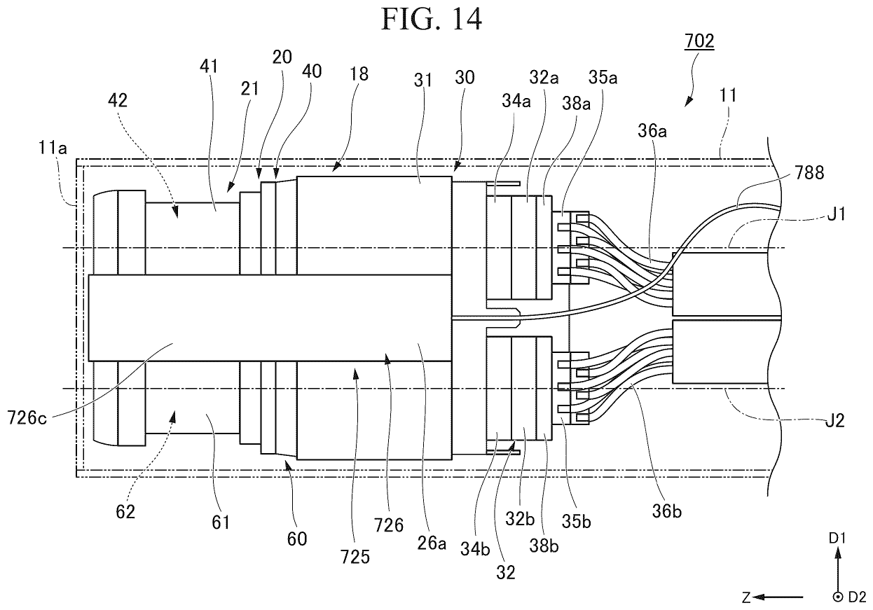

FIG. 14 is an external view of the optical unit 18 of this embodiment when seen from a first side (the +D2 side) in the second direction. In the following description, the same components as those in the first embodiment described above are designated by the same reference numerals, and the description thereof will be omitted. As shown in FIG. 14, an endoscope 702 of this embodiment includes the optical unit 18, a heat dissipation member 725, the imaging unit 30, and a ground cable 788.

The heat dissipation member 725 includes a first heat dissipation member 726 and a second heat dissipation member (not shown). The first heat dissipation member 726 is disposed closer to a first side (the +D2 side) in the second direction D2 than the objective lens unit 20 and the imaging unit 30. The first heat dissipation member 726 has a first fixing portion 26a and a first contact portion 726c. The first fixing portion 26a is in contact with the imaging element holding part 31.

The first contact portion 726c has a triangular prism shape that extends from an end portion of the first fixing portion 26a on the object side (the +Z side) toward the object side. In this embodiment, the end portion of the first contact portion 726c on the object side passes through an opening (not shown) in the tip end surface 11a and is located closer to the object side than the tip end surface 11a. Thus, an end portion of the heat dissipation member 725 on the object side is exposed to the outside of the endoscope 702. Other configurations of the first contact portion 726c are similar to other configurations of the first contact portion 26c of the first embodiment described above.

Although not shown in the drawing, the second heat dissipation member is disposed closer to the second side (the −D2 side) in the second direction D2 than the objective lens unit 20 and the imaging unit 30. The second heat dissipation member has a second fixing portion (not shown) and a second contact portion (not shown). The first fixing portion is in contact with the imaging element holding part 31. The second contact portion has a triangular prism shape that extends from an end portion of the second fixing portion on the object side (the +Z side) toward the object side. Like the first contact portion 726c, the end portion of the second contact portion on the object side passes through an opening (not shown) in the tip end surface 11a and is located closer to the object side than the tip end surface 11a. Thus, an end portion of the heat dissipation member 725 on the object side is exposed to the outside of the endoscope 702. Other configurations of the second fixing portion and the second contact portion of this embodiment are similar to other configurations of the second fixing portion and the second contact portion 27c of the first embodiment described above.

The ground cable 788 is a cable that extends from the heat dissipation member 725 toward the base end side (the −Z side). The ground cable 788 is electrically conductive. Although not shown in the drawing, in this embodiment, the ground cable 788 is connected to a ground line of the processor 3 (refer to FIG. 1). Although not shown in the drawing, the ground line of the processor 3 is grounded. Therefore, the heat dissipation member 725 is grounded via the ground cable 788 and the ground line. The ground cable 788 may be connected to a conductive wire that is grounded among the conductive wires of the connector part 8a (refer to FIG. 1). The ground cable 788 may also be connected to a metal exterior such as the tip end portion 11, the rigid tube portion 13, or the like.

According to this embodiment, the end portion of the heat dissipation member 725 on the object side (the +Z side) is exposed to the outside of the endoscope 702, and the heat dissipation member 725 is grounded. Therefore, static electricity generated in the vicinity of the tip end portion 11, particularly in the vicinity of the tip end surface 11a can be transmitted to the ground via the heat dissipation member 725 and the ground cable 788. Thus, it is possible to suppress the static electricity from being transmitted to the imaging element 32. Therefore, it is possible to suppress the imaging element 32 from being damaged by the static electricity, and also possible to suppress the image signal converted by the imaging element 32 from deteriorating. Therefore, the stability of the operation of the endoscope 702 can be improved.

Although the embodiments of the present invention has been described above, each of the configurations and combinations thereof in the embodiments is merely an example, and addition, omission, substitution, and other modifications of the configurations are possible without departing from the spirit of the present invention. Furthermore, the present invention is not limited to the embodiments.

The number of imaging elements included in the endoscope may be one, or three or more. When there is one imaging element, the imaging element converts the optical images of the subject formed by each of the first objective lens unit and the second objective lens unit into the image signals, and thus the amount of heat generated in the imaging element tends to be large. However, in the embodiment of the present invention, the heat generated in the imaging element can be transferred to the lens frame via the heat dissipation member, and thus it is possible to suppress the temperature of the imaging element from becoming too high.

The heat dissipation member only needs to be in contact with either the first lens frame or the second lens frame. Even in this case, since the heat generated in the imaging element can be transferred to the lens frame via the heat dissipation member, it is possible to suppress the temperature of the imaging element from becoming too high.

Furthermore, as long as the heat dissipation member is in contact with the lens frame, the shape of the heat dissipation member, particularly the contact portion, is not limited to this embodiment, and for example, the contact portion may have other shapes, such as a circular shape or a rectangular shape, when seen in the optical axis direction. The endoscope may also include three or more heat dissipation members.

What is claimed is:

1. An endoscope comprising:
a lens frame configured to hold each of a first objective optical system and a second objective optical system and to extend in an optical axis direction;
an imaging unit having an imaging element; and
a heat dissipation member in contact with each of the lens frame and the imaging unit.

2. The endoscope according to claim 1, wherein the lens frame includes a first lens frame that holds the first objective optical system, and a second lens frame that holds the second objective optical system,
the imaging unit has at least one imaging element, and
at least a part of the heat dissipation member is disposed between the first lens frame and the second lens frame and is in contact with at least one of the first lens frame and the second lens frame.

3. The endoscope according to claim 2, wherein the heat dissipation member is in contact with both the first lens frame and the second lens frame.

4. The endoscope according to claim 1, further comprising a holding frame configured to accommodate the first lens frame, the second lens frame, and the imaging unit,
wherein the heat dissipation member and the holding frame are different members.

5. The endoscope according to claim 4, wherein a thermal conductivity of the heat dissipation member is greater than a thermal conductivity of the holding frame.

6. The endoscope according to claim 1, wherein the first lens frame and the second lens frame are disposed with an interval therebetween in a first direction intersecting the optical axis direction, and
when seen in the first direction, at least a part of the heat dissipation member overlaps each of the first lens frame and the second lens frame.

7. The endoscope according to claim 1, wherein the imaging unit has an imaging element holding part that holds the imaging element,
the imaging element holding part is made of a resin, and
the heat dissipation member is in contact with the imaging element holding part.

8. The endoscope according to claim 1, further comprising a heat transfer member disposed closer to a base end side than the imaging element,
wherein the heat transfer member is in contact with the imaging unit.

9. The endoscope according to claim 1, wherein the heat dissipation member has a contact portion that is in contact with the lens frame,
a straight line that intersects a central axis of each of the first lens frame and the second lens frame and that extends in a first direction intersecting the optical axis direction is defined as a first virtual line, and
in a second direction that is a direction perpendicular to both the optical axis direction and the first direction, a dimension of a portion of the contact portion in the first direction that is farthest from the first virtual line in the second direction is larger than a dimension of a portion of the contact portion in the first direction that is closest to the first virtual line.

10. The endoscope according to claim 9, wherein the contact portion has a first portion that extends in the second direction and second portions that extend in the first direction from both ends of the first portion in the second direction.

11. The endoscope according to claim 9, wherein when seen in the optical axis direction, the contact portion has a substantially triangular shape that protrudes toward the first virtual line.

12. The endoscope according to claim 9, wherein the heat dissipation member includes a first heat dissipation member disposed closer to a first side in the second direction than the first virtual line, and a second heat dissipation member disposed closer to a second side in the second direction than the first virtual line,
the first heat dissipation member has a first contact portion that is in contact with the lens frame,
the second heat dissipation member has a second contact portion that is in contact with the lens frame, and
when seen in the optical axis direction, each of the first contact portion and the second contact portion has a substantially triangular shape that protrudes toward the first virtual line.

13. The endoscope according to claim 9, wherein when seen in the optical axis direction, the contact portion has a V-shape that widens in the first direction as it moves away from the first virtual line in the second direction.

14. The endoscope according to claim 9, wherein the first lens frame has a first moving frame that is movable in the optical axis direction, and a first holding portion that movably holds the first moving frame,
the second lens frame has a second moving frame that is movable in the optical axis direction, and a second holding portion that movably holds the second moving frame,
each of the first moving frame and the second moving frame holds at least one lens,
a magnet is mounted in each of the first moving frame and the second moving frame,
a coil is mounted on each of the first holding portion and the second holding portion, and
each of the first moving frame and the second moving frame moves in the optical axis direction by passing a current through the coil.

15. The endoscope according to claim 14, wherein the heat dissipation member is made of a magnetic material.

16. The endoscope according to claim 14, wherein the heat dissipation member is disposed on only a first side in the second direction with respect to the first virtual line.

17. The endoscope according to claim 1, further comprising a heat-conducted member and a heat transfer member configured to connect the heat dissipation member and the heat conductive member.

18. The endoscope according to claim 1, wherein an end portion of the heat dissipation member on an object side is exposed to an outside of the endoscope, and
the heat dissipation member is grounded.
